(19) United States
(12) Patent Application Publication
(10) Pub. No.: US 2005/0164168 A1
(43) Pub. Date: Jul. 28, 2005

(54) Method for the rapid diagnosis of infectious disease by detection and quantitation of microorganism induced cytokines

(76) Inventors: Malford E. Cullum, Grayslake, IL (US); Gerald H. Mazurek, Dunwoody, GA (US)

Correspondence Address: NAVAL MEDICAL RESEARCH CENTER;ATTN: (CODE 00L), 503 ROBERT GRANT AVENUE, SILVER SPRING, MD, 20910-7500, US

(21) Appl. No.: 10/855,325

(22) Filed: May 28, 2004

Related U.S. Application Data
(63) Continuation-in-part of application No. 10/809,877, filed on Mar. 26, 2004.
(60) Provisional application No. 60/457,940, filed on Mar. 28, 2003.

(51) Int. Cl. C12Q 1/70; G01N 33/53; G01N 33/569; G01N 33/554

(52) U.S. Cl. 435/5; 435/7.22; 435/7.32

(57) ABSTRACT

The inventive subject matter relates to a competitive method for the diagnosis of latent infectious disease, such as Mycobacterium tuberculosis, by estimating, the concentration of cytokine, such as interferon-gamma produced by stimulated immune cells, collected from whole blood, by Fluorescence Polarization (FP), Fluorescence Resonance Energy Transfer (FRET) or Fluorescence Lifetime (FLT) due to antibody-cytokine interaction or by dimerization of the cytokine.

### CROSS REFERENCE TO RELATED APPLICATION

This application is a Continuation in Part of U.S. application No. 10/809,877 filed Mar. 26, 2004 that claims priority to Provisional application No. 60/457,940 filed Mar. 28, 2003.

### FIELD OF THE INVENTION

The inventive subject matter relates to a competitive fluorescence method for diagnosing infectious disease, including tuberculosis, by quantitating interferon-gamma and other cytokine concentration from stimulated immune cells collected from whole blood. The method contemplates the use of FLT, FRET or FP.

### BACKGROUND OF INVENTION

Infectious diseases caused by pathogenic bacteria and viruses, is a serious health concern cause in the United States and other developed nations. Infectious disease morbidity and mortality is especially important in developing nations with poor health care resources.

One of the most important infectious diseases is tuberculosis. Tuberculosis remains one of the world's deadliest of diseases. The World Health Organization estimates that each year more than 8 million new cases occur and approximately 3 million persons die from the disease (1). While the majority of M. tuberculosis infections are kept in check by the host's immune defenses and remain latent, some latent infections progress to active and contagious disease. The number of persons with latent TB infection (LTBI) in the United States is estimated to range from 10 million to 15 million, and many cases of active tuberculosis arise from this pool of infected persons (2).

Until recently, skin testing with purified protein derivative (PPD) of tuberculin was the only practical way of detecting latent M tuberculosis infections. In the United States, the tuberculin skin test (TST) is used as an initial screening test for both latent infection and active tuberculosis (3). A positive TST is indicative of an increased risk of subsequently developing, or currently having, active tuberculosis (4). However, despite its widespread use and a large body of data on its standardization, the TST is subject to significant variations and limitations. False-positive TST responses may result from contact with non-tuberculosis Mycobacterium (NTM) that share common antigens with M tuberculosis, or may result from prior BCG vaccinations (5-8). Errors in placement and reading of the TST can also give false-positive results. A multitude of conditions may blunt the response to tuberculin, most notably HIV-associated immunosuppression; the largest cause of erroneous TST results lies with the subjective nature of placement and reading of the test (1, 9). Digit-preference (for example, rounding measures of TST in duration to the nearest multiple of 5 mm) and interpretation bias significantly affect TST results (10).

Identifying persons with LTBI is crucial to the goal of tuberculosis elimination, because the development of active tuberculosis in these persons can effectively be prevented with treatment, thereby stopping further spread of disease (11). The Institute of Medicine, in a report published in 2000, gave high priority to the development of tools with which to identify persons with LTBI and those at greatest risk of developing active tuberculosis (12).

Cytokines play an important role in the regulation of T cells. Some cytokines, such as tumor necrosis factor (TNF) or Interferon gamma (IFN-γ) lead to cell mediated immune responses against infectious agents. Other cytokines (e.g. Interleukins 2, 4, 12) are critical in the development of humoral immune responses (13).

Interferon gamma (IFN-γ), is a 21 to 24-kDa glycoprotein synthesized by activated T cells and natural killer cells, forming a homodimer once secreted (14-16) and possesses antiproliferative and antiviral activity. IFN-γ is also a potent activator of mononuclear phagocytes, increasing their ability to destroy intracellular microorganisms and tumor cells and causes the induction of major histocompatibility proteins. As a result of these properties, IFN-γ is an important cytokine in the immune response to bacterial infection, such as Mycobacterium tuberculosis.

Recently, commercially available methods exist for the detection of M. tuberculosis infection. A commercial assay, QuantiFERON®-TB, (QFT) (17) offers a method to detect latent disease, requiring only a single patient, based on quantification of IFN-γ in blood samples. The QFT is an in vitro whole blood gamma interferon (IFN-γ) release assay, and is capable of assessing the response to multiple antigens simultaneously, and does not boost anamnestic immune responses, such as in TST. Interpretation of the QFT is less subjective than that of the TST and the QFT appears less affected than TST by prior BCG vaccination. A Food and Drug Administration (FDA) panel recently approved the QFT “as an aid for the detection of M. tuberculosis infection” based primarily on results from an evaluation by Centers for Disease Control and Prevention (CDC) (18). For the QFT, the amount of IFN-γ released is determined by an enzyme linked immunosorbent assay (ELISA). The ELISPOT is a similar in vitro test that quantifies the number of lymphocytes that produce IFN-γ instead of measuring the total amount of IFN-γ released in response to tuberculosis antigens.

As previously mentioned, in the QFT the concentration of IFN-γ is determined by an ELISA (17). A limitation of ELISA, however, is that it suffers intrinsically from much higher non-specific binding being a heterogeneous (surface binding) assay rather than homogeneous (direct ligand-receptor binding in aqueous solution) fluorescence polarization (FP) type assay. Furthermore, ELISA requires numerous wash steps and lengthy incubation steps, often requiring 3 to 4 hours to complete the assay. The ELISA method is also dependent on clear optical path. Cloudy solutions are often not amenable to ELISA conducted in liquid phase or with particulate matter.

Results of animal and human studies of the QFT conducted worldwide have been encouraging (19-21). Because there is no “gold standard” for confirming LTBI in humans (2, 1), we compared results of QFT to TST in the initial CDC multi-center study of QFT (21), and then used multivariate analysis to identify subject-related and test-related factors associated with discordance between the tests. Overall agreement between QFT and TST was 83% when the tests were performed simultaneously using PPD from M. tuberculosis in both tests (21). Agreement between the tests improved to 88% when analysis was restricted to people being screened for LTBI who had no history of BCG vaccination. Agreement improved further, to 91%, when people with evidence of NTM infection were excluded. Three major factors were associated with test discordance: prior BCG vaccination, evidence of immunity to NTM, and digit-preference in reading the TST at some study sites. BCG vaccinated individuals were 6 times more likely to have a positive TST but a negative QFT than unvaccinated individuals. Individuals with evidence of immunity to NTM by QFT were 3 times more likely to have a positive TST but negative QFT than individuals without such immunity. Discordance at some sites was 4 times that at others, and sites with the greatest discordance had the greatest digit-preference for 10 and 15 mm TST readings.

Pottumarthy et al. found a similar level of agreement between the two tests in a study involving New Zealand health care workers and immigrants (20) whereas Streeton et al. reported a somewhat better concordance (98%) for persons with no known exposure and a negative TST, and 90% for untreated TST reactors (21). The level of agreement found between the QFT and TST in the CDC study is similar to the level of agreement found when TSTs using two different commercial PPD preparations (Tubersol and Aplisol) were administered simultaneously (22, 23).

In the past, differentiation of immunity to M. tuberculosis from immunity to NTM required placement of two skin tests, one using PPD from M. tuberculosis, the other using PPD from the NTM of interest (3, 24). The larger reaction indicated the infecting organism while the smaller reaction was considered secondary to cross reactivity to conserved antigens. The QFT assay allows differentiation by determining which antigens, those from M tuberculosis or those from NTM, induce greater IFN-γ production. Theoretically this does not eliminate the problem of differentiating LTBI from prior BCG vaccination. However, genetic studies have identified M. tuberculosis antigens that are not shared with other mycobacteria, and thus may be more specific for immunity to M. tuberculosis than PPD. ESAT-6 is a protein found in PPD from M. tuberculosis but not BCG or other common NTM. Immune responses to ESAT-6 are detected in 60 to 80% of tuberculosis patients (17). Lalvani, et al studied 50 people with recent TB exposure and found that response to ESAT-6 (measured by ELISPOT) was more closely associated with recent TB exposure than TST (25). Preliminary studies indicate that inclusion of recombinant M. tuberculosis-specific antigens, such as ESAT-6, in the QFT assay improves specificity. Of ten recombinant antigens screened during the initial CDC QFT study, ESAT-6 showed the greatest promise (21). Additional recombinant antigens have been identified as potential diagnostic reagents in preliminary studies (26, 27) and are available in adequate amounts for evaluation in this study. Like ESAT-6, these antigens are produced by clinical strains of M. tuberculosis but not by BCG.

A new version of QFT has been developed which involves collection of 1 mL of blood into vacutainer tubes, which already contain the TB-specific antigens. This version of the test has been termed QuantiFERON®-TB-3G (QFT-3G). The blood tubes are simply filled by venipuncture, mixed and incubated upright at 37° C. overnight. The next day, the plasma is tested for IFN-γ by ELISA. The ELISA procedure used for the QFT-3G test is the same as the assay used for other TB-specific antigens. The QFT-3G test format practically removes the 12 hour logistic problem of the original QFT test, involves far less labor (and thus will be cheaper to perform), requires less blood, is easily automated, and has proven to be more sensitive in initial, small-scale, clinical studies.

Previous studies of the QFT and tuberculosis have been limited because children have not been included as study subjects and the association between QFT and the risk of tuberculosis has not been examined. While there is no evidence to suggest that QFT will perform differently in children as compared to adults, this question has not been addressed. Studies to assess the association between QFT results and risk of tuberculosis may be prohibitively large and lengthy. This is because few people (about 15%) have discordant TST and QFT results, only 10% of people with LTBI progress to active tuberculosis, and infected people at increased risk of tuberculosis activation are treated for LTBI. However, if QFT is shown to be more closely associated with recent exposure than TST, it may predict more accurately who will benefit from treatment for LTBI because people with recent infection have the greatest risk of developing tuberculosis (4).

Therefore, because of the pivotal role played by IFN-γ in the response to M. tuberculosis infection, the measurement of IFN-γ release can be used as an alternative to tuberculin skin testing (TST) in detecting these infections (28, 29). However, because of the time and expense in quantifying cytokines from blood samples, using currently available methods, development of improved cytokine detection and quantification assays are needed, over ELISA and other available methods.

Fluorescent polarization (FP) technology is capable of rapid, real-time, sensitive evaluation of fluid phase antigens with high specificity (30, 31). The basis for FP assays was initially described by Perrin (32), and Danliker (33), and patented by Abbott Laboratories (34). FP based assays are predicated on the principle that polarized incident visible or ultraviolet light that illuminates a fluorochrome causes subsequent polarized fluorescence with emission at a longer wavelength. However, molecules in solution are capable of rotation. Therefore, polarized light striking a fluorescent molecule loses polarization due to rotation of the molecule. Solutions containing slower turning, large molecule-fluorochrome complexes tends to stay polarized longer verses situations where smaller labeled molecules are present. In order to accommodate molecules of different sizes (up to 10⁷ kDa molecular weight), different fluorochromes can be selected (35).

Combining fluorochrome-labeled antigen or peptide with antibody results in an increase in FP, as measured in arbitrary millipolarization (mP) units. The smaller the fluorescent antigen, the greater the increase in mP units that is measured upon binding to its corresponding antibody, since mP depends upon the partial specific volume (approximate molecular weight in solution) of the labeled substance. The dependence is non-linear but is describable in a Perrin equation (36).

FP antigen-antibody binding assays are theoretically simple to perform, requiring only the mixing liquid test samples with fluorescent reagent. In a rapid diagnostic format, essentially two FP readings are necessary; a base-line reading and a reading after a specified time. The FP value increases as binding of flurorescently labeled ligand occurs. For example, the difference in FP between a fluorescent antigen of 10 kDa initially and the fluorescent complex consisting of it and IgG, for example, results in a measurable association using less than saturating antibody concentrations (37).

A distinct advantage of FP technology is that FP assays can be designed to be much more rapid than the 3 to 4 hours required in a typical ELISA with the FP assay requiring little if any wash steps. FP technology is also immune to cloudiness, particles, blood cells (inner filter effects) and does not require a clear optical path. Therefore, FP detection technology, unlike ELISA, can accommodate cloudy solutions, such as blood samples, with minimal clarifying preparatory steps (38). FP assays can also be designed to accommodate significant variation in pH in fluid samples by utilizing different pH-independent fluorochromes (38, 39).

Currently, FP assays are in used to measure different types of binding reactions, to follow proteolytic reactions and to measure various enzymatic or receptor binding reactions (40, 41). In clinical settings, FP assays are used to measure the level of drugs, hormones or antibiotics in blood plasma (42). ELISA, RIA and immunoprecipitation assays for the measurement of antibody to infectious agents in serum are the most accepted methods for detection prior infection of viral or bacterial (37, 43, 44). Because of its advantages over other detection methods, FP is well-suited as a diagnostic tool for analyzing formerly difficult to evaluate samples such as oral fluids and saliva, in addition to serum, for the quantitative assessment of specific antibody, diagnostic markers, drugs, chemicals and infectious or biohazardous agents.

### SUMMARY OF THE INVENTION

Current methods for the diagnosis of latent Mycobacteria tuberculosis, and other infectious diseases, are inadequate. TST, the method used over the last several decades, suffers from being an in vivo test requiring multiple patient visits to administer and then read the results. The TST assay also causes a boost phenomenon causing false positive results upon subsequent tests. Newer, commercially available methods, employ in vitro procedures to measure interferon-gamma responses by the patients blood cells and require only single laboratory visits without any boost phenomenon. However, these methods suffer from the requirement for laborious and time consuming laboratory analysis procedures, such as ELISA, for the final quantitation of cytokine levels. Therefore, a laboratory cytokine quantitation method that is rapid, easy to conduct and sensitive is important for accurate, routine diagnosis of latent Mycobacterium infection. Therefore, this invention relates to the detection and quantitation of cytokines in blood samples collected from patients suspected of being previously exposed and infected with infectious viral and bacterial agents, such as M. tuberculosis.

An object of the invention is the detection and quantitation of interferon-gamma and other cytokines, by fluorescent polarization (FP), fluorescence lifetime (FLT) analysis or fluorescence resonance energy transfer (FRET).

A further object, is the quantitation of interferon-gamma and other cytokines, by FP, FLT or FRET where interferon-gamma has been induced by in vitro stimulation of T cells by specific antigen.

A still further object of the invention is the detection and quantitation of interferon-gamma by measuring the competitive binding by interferon-specific antibody by FP, FLT or FRET.

Another object of the invention is the detection and quantitation of interferon-gamma or other cytokine by measuring the competitive dimerization of interferon-gamma or cytokine by FP, FLT or FRET.

Still another object of the invention is the detection and quantitation of interferon-gamma or other cytokine by measuring the binding of interferon-gamma or other cytokine, by FP, FLT or FRET, to antigen presenting cells from immune patients.

These and other objects of the invention are accomplished by employing FP, FLT or FRET in a competitive assay.

### BRIEF DESCRIPTION OF THE DRAWINGS

FIG. 1. Diagram showing expected change in polarization at various concentrations of competitive cytokine. For complete description, see prophetic example 1.

### DETAILED DESCRIPTION OF THE PREFERRED EMBODIMENTS

Fluorescent polarization holds great promise as an assay method for frequent, routine, cost-effective means of obtaining quantitation of cytokines and detection of immune cells isolated from blood from potentially infected patients. Fluorescent molecules emit polarized fluorescence when they absorb polarized light at a specific wavelength. However, inherent in molecules in solution is their tendency to rotate. When polarized light strikes the molecules in solution, the emitted light does not remain polarized because the molecule is rotating rapidly in solution. The rate of rotation, however, is dependent on the size of the molecule. Therefore, an antigen/antibody complex will have an inherently slower rate of rotation causing more of the polarized fluorescence to be emitted in the same plane as the incident light. The polarization-based readouts are less susceptible to environmental interferences, such as pH, cloudiness, and particles in the assay compartment than other light-based assays. FP is fundamentally and theoretically different from most color based photometric techniques and gains its analytical advantage by this intrinsic property of being independent of concentration. The current application utilizes FP technology in a competitive assay to quantitate infection induced cytokines or immune cells in patient blood samples for the diagnosis of infectious diseases such as tuberculosis.

It is known that interferon-gamma (IFN-γ) plays an important role in the immune response to Mycobacterium tuberculosis, as well as other bacterial infectious organisms. Therefore, detection of induction IFN-γ in serum or plasma produced by immune cells from infected patients would be an important diagnostic tool. Detection can be obtained by either direct measure of interferon-gamma using specific antibody in a competitive fluorescent polarization immunoassay (CFIPA) or by detection of homodimerization of interferon-gamma by dimerization induced fluorescence polarization (DIFP). In either case, detection and quantitation will be down to less than 11 pg/ml.

Prophetic Example 1

### Competitive FP assay for measuring interferon-gamma in serum or plasma by competitive assay using interferon-gamma specific antibody

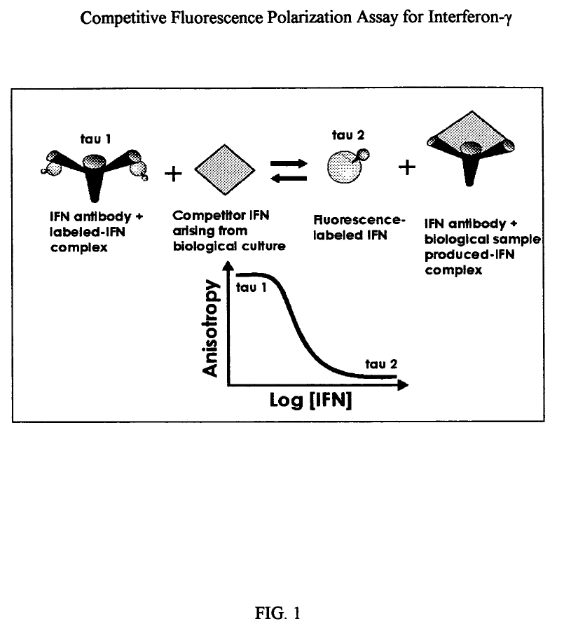

In the current application we disclose a competitive fluorescence polarization immunoassay (CFPIA) that can measure IFN-γ concentrations in a fraction of the time required for ELISA. To measure IFN-γ concentrations, the assay will be conducted as illustrated, generally, in FIG. 1. Reagents include: 1) the use of fluorescently labeled IFN-γ which can be made from commercially available reagents, and 2) the use of a monoclonal IFN-γ specific antibody. The labeled IFN-γ will produce polarized fluorescence if appropriately aligned when excited by polarized light (of the appropriate wavelength). However, only a small portion of the fluorescence will be captured by the polarization detector because IFN-γ is a small molecule that changes its orientation rapidly and becomes depolarized. Encumbering the IFN-γ, for example by attaching an antibody, will increase the amount of fluorescence detected. The subsequent addition of unlabeled IFN-γ will decrease the amount of polarized fluorescence detected proportionate to the amount of unlabeled IFN-γ added. The assay is conducted by the following steps:
a. Collecting whole blood samples (with anti-clotting agent, e.g. heparin) from patients suspected of viral, protozoa or bacterial infection, such as M. tuberculosis and containing potential immune (i.e. primed) T cells;
b. Adding viral, protozoa or bacterial antigen (such as M. tuberculosis antigen) to whole blood sample;
c. Incubate the whole blood/antigen culture mixture as long as overnight (The immune (i.e. primed) cells are induced to produce cytokine by their recognition of specific antigen in Step (b));
d. Collect serum or plasma fractions and prepare dilutions. Include in the sample preparation a standard curve of serum with known concentrations of cytokine (interferon-gamma) or specific fragments of interferon-gamma;
e. Add to the serum or plasma dilutions interferon-gamma fluorescently labeled probe at about 1 nM;
f. Add antibody (polyclonal or monoclonal) specific to interferon-gamma to each of the dilutions at appropriate antibody dilution;
g. Measure the change in fluorescence polarization of the serum dilutions;
h. Graphically compare the concentration of test sample dilutions to standard curve to determine concentration of cytokine (interferon-gamma).

The assay mixture would require 0.01 ml plasma (20 pg/ml IFN-γ, or about 2 pM) up to 0.1 ml plasma. The intrinsic blood plasma or serum fluorescence can be suppressed by the addition of fluorescent quenchers, if necessary.

### Prophetic Example 2

### Competitive FP assay for measuring IFN-γ in serum or plasma by competitive assay measuring change in fluorescence polarization due to homodimerization

IFN-γ is a 21 to 24 kDa protein, synthesized by T cells and natural killer cells, that quickly tends to form homodimers in solution. The concentration of interferon-gamma can be detected, therefore, by the concentration of interferon-gamma can be detected by the change in fluorescence polarization induced by dimerization (DIFP). The assay is conducted by the following steps:
a. Collecting whole blood samples from patients suspected of viral, protozoa or bacterial infection, such as M. tuberculosis and containing potential immune T cells;
b. Adding viral, protozoa or bacterial antigen (such as M. tuberculosis antigen) to whole blood sample;
c. Incubate the whole blood/antigen culture mixture as long as overnight;
d. Collect serum or plasma fractions and prepare dilutions. Include in the sample preparation a standard curve of serum with known concentrations IFN-γ;
e. Add to the serum or plasma dilutions IFN-γ fluorescently labeled probe at about 1 nM. Alternatively, peptide fragments, that are fluorescently labeled can be used as a probe;
f. Measure the change in fluorescence polarization of the serum dilutions due to dimerization;
g. Graphically compare the concentration of test sample dilutions to standard curve to determine concentration of cytokine (IFN-γ).

A further aspect of the invention is the detection and quantitation by fluorescence polarization, fluorescence life-time (FLT) and fluorescence resonance energy transfer (FRET). If FLT is used, then detection is dependent on a change in fluorescence life-time. If FRET is used, then detection is by sensitized fluorescence of the acceptor or by quenching of donor fluorescence or by fluorescence depolarization.

A still further aspect of the invention is that different fluorochromes, added to the cytokine (e.g. interferon-gamma or specific M. tuberculosis probes), can be utilized in order to optimize results, including the incorporation of pH-independent fluorochromes. The fluorochromes that are included as an aspect of the invention include: 7-AAD, Acridine Orange, Alexa 488, Alexa 532, Alexa 546, Alexa 568, Alexa 594, Aminonapthalene, Benzoxadiazole, BODIPY 493/504, BODIPY 505/515, BODIPY 576/589, BODIPY FL, BODIPY TMR, BODIPY TR, Carboxytetramethylrhodamine, Cascade Blue, a Coumarin, Cy2, CY3, CY5, CY9, Dansyl Chloride, DAPI, Eosin, Erythrosin, Ethidium Homodimer II, Ethidium Bromide, Fluorescamine, Fluorescein, FTC, GFP (yellow shifted mutants T203Y, T203F, S65G/S72A), Hoechst 33242, Hoechst 33258, IAEDANS, an Indopyras Dye, a Lanthanide Chelate, a Lanthanide Cryptate, Lissamine Rhodamine, Lucifer Yellow, Maleimide, MANT, MQAE, NBD, Oregon Green 488, Oregon Green 514, Oregon Green 500, Phycoerythrin, a Porphyrin, Propidium Iodide, Pyrene, Pyrene Butyrate, Pyrene Maleimide, Pyridyloxazole, Rhodamine 123, Rhodamine 6G, Rhodamine Green, SPQ, Texas Red, TM, TOTO-1, TRITC, YOYO-1, vitamin B12, flavin-adenine dinucleotide, and nicotinamide-adenine dinucleotide.

### REFERENCES

1. Diagnostic Standards and Classification of Tuberculosis in Adults and Children. This official statement of the American Thoracic Society and the Centers for Disease Control and Prevention was adopted by the ATS Board of Directors, July 1999. This statement was endorsed by the Council of the Infectious Disease Society of America, September 1999. Am J Respir Crit Care Med Apr 2000; 161(4 Pt 1):1376-1395.

2. Screening for tuberculosis and tuberculosis infection in high-risk populations. Recommendations of the Advisory Council for the Elimination of Tuberculosis. MMWR Recomm Rep. Sep. 8, 1995; 44(RR-11):19-34.

3. Judson F N, Feldman R A. Mycobacterial skin tests in humans 12 years after infection with Mycobacterium marinum. Am Rev Respir Dis. May 1974; 109(5):544-547.

4. Mandell G L, Bennett J E, Dolin R, eds. Mandell, Douglas, and Bennetl's Principles and Practice of Infectious Diseases. 5 ed. Philadelphia, Pa.: Churchill Livingstone; 2000.

5. Judson F N, Feldman R A. Mycobacterial skin tests in humans 12 years after infection with Mycobacterium marinum. Am Rev Respir Dis. May 1974; 109(5):544-547.

6. Snider D E, Jr. Bacille Calmette-Guerin vaccinations and tuberculin skin tests. Jama. Jun 21, 1985; 253(23):3438-3439.

7. von Reyn C F, Williams D E, Horsburgh C R, Jr., et al. Dual skin testing with Mycobacterium avium sensitin and purified protein derivative to discriminate pulmonary disease due to M. avium complex from pulmonary disease due to Mycobacterium tuberculosis. J Infect Dis. Mar 1998; 177(3):730-736.

8. Andersen, P., Munk, M. E., Pollock, J. M., Doherty, T. M. (2000) Specific immune based diagnosis of tuberculosis. Lancet 356 (9235):1099-1104.

9. Huebner R E, Schein M F, Bass J B, Jr. The tuberculin skin test. Clin Infect Dis. Dec 1993; 17(6):968-975.

10. Pottumarthy S, Morris A J, Harrison A C, Wells V C. Evaluation of the tuberculin gamma interferon assay: potential to replace the Mantoux skin test. J Clin Microbiol. Oct 1999; 37(10):3229-3232.

11. Blumberg H M, Burman W J, Chaisson R E, et al. American Thoracic Society/Centers for Disease Control and Prevention/infectious Diseases Society of America: treatment of tuberculosis. Am JRespir Crit Care Med. Feb. 15, 2003; 167(4):603-662.

12. Geiter L, ed. Ending Neglect: The Elimination of Tuberculosis in the United States. Washington D.C.: National Academy Press; 2000.

13. Paul, W. E., and Seder, R. A (1994) Lymphocyte responses and cytokines. Cell 76: 241.

14. Yip, Y. K., Barrowcloughn, B. S., Urban, C., Vilcek, J. (1982) Purification of two subspecie of human gamma (immune) interferon. Proc. Natl. Acad. Sci USA 79: 1820.

15. Rinderknecht, E., O'Connor, B. H., Rodrigues, H., Natural human interferon-γ. Complete amino acid sequence and determination of sites of glycosylation. J. Biol. Chem. (1984) 259: 6790.

16. Zlateva, T., Boteva, R., Salvato, B., Tsanev, R. (1999) Factors affecting the dissociation and aggregation of human interferon gamma. Int. J. Biol. Macromol. 26(5): 357-362.

17. QuantiFERON-TB, The Whole Blood IFN-gamma TEST, An Aid to Detect M. tuberculosis Infection. Melbourne, Australia: Cellestis; 2002.

18. CDC. Guidelines for using the QuantiFERON®-TB test for diagnosisng laten Mcobacterium tuberculosis infection. MMWR 2003, 52(No. RR-02): 15-18.

19. Wood P R, Comer L A, Rothel J S, et al. Field comparison of the interferon-gamma assay and the intradermal tuberculin test for the diagnosis of bovine tuberculosis. Aust Vet J. Sep 1991;68(9):286-290.

20. Pottumarthy S, Morris A J, Harrison A C, Wells V C. Evaluation of the tuberculin gamma interferon assay: potential to replace the Mantoux skin test. J Clin Microbiol. Oct 1999; 37(10):3229-3232.

21. Mazurek G H, LoBue P A, Daley C L, et al. Comparison of a whole-blood interferon gamma assay with tuberculin skin testing for detecting latent Mycobacterium tuberculosis infection. Jama. Oct. 10, 2001; 286(14):1740-1747.

22. Villarino M E, Burman W, Wang Y C, et al. Comparable specificity of 2 commercial tuberculin reagents in persons at low risk for tuberculous infection. Jama. Jan. 13, 1999; 281(2):169-171.

23. Villarino M E. Personal communication.

24. Chaparas S. Immunologically Based Diagnostic Tests with Tuberculin and Other Mycobacterial Antigens. In: Kubica G, Wayne L, eds. The Mycobacteria: A Source Book. New York: Marcel Dekker, Inc.; 1984: 195-220.

25. Lalvani, A., Pathan, A. A., Durkan, H., et al (2001) Enhanced contact tracing and spatial tracking of Mycobacterium tuberculosis infection by enumeration of antigen specific T cells. Lancet 357(9273): 2017-2021.

26. van Pinxteren L A, Ravn P, Agger E M, Pollock J, Andersen P. Diagnosis of tuberculosis based on the two specific antigens ESAT-6 and CFP10. Clin Diagn Lab Immunol. Mar 2000; 7(2):155-160.

27. Brock I, Munk M E, Kok-Jensen A, Andersen P. Performance of whole blood IFN-gamma test for tuberculosis diagnosis based on PPD or the specific antigens ESAT-6 and CFP-10. Int J Tuberc Lung Dis. May 2001; 5(5):462-467.

28. Andersen P, Munk M E, Pollock J M, Doherty T M. Specific immune-based diagnosis of tuberculosis. Lancet. Sep. 23, 2000; 356(9235):1099-1104.

29. Rothel, J. S., Jones, S. L., Corner, L. A., Cox, J. C., Wood, P. R. (1990) A sandwich enzyme immunoassay for bovine interferon-gamma and its use for the detection of tuberculosis in cattle. Aust. Vet. J. 67(4): 134-137.

30. Kowski T, Wu J: Fluorescence polarization is a useful technology for reagent reduction in assay miniaturization, Comb Chem High Throughput Screen 2000; 3:437-44.

31. ONR: Future Naval Capabilities: Warfighter Protection, Washington, D.C., Department of the Navy: Science and Technology Lead, 1992.

32. Perrin F. Polarization de la lumiere fluorescence. Vie moyenne de molecules dans l'etat excite. J Phys Radium. 1926; 7:390.

33. Dandliker W, Feigen G. Quantification of the antigen-antibody reaction by the polarization of fluorescence. Biochem Biophys Res Comm. 1961; 5(4):299-304.

34. Popelka S, inventor; Abbott Laboratories, assignee. Apparatus and method for fluorescence polarization instrument: Pat. No. 4,516,856, 1985.

35. Terpetschnig E, Szmacinski H, Lakowicz J: Fluorescence polarization immunoassay of a high-molecular-weight antigen based on a long-lifetime Ru-ligand complex, Anal Biochem 1995; 227:140-7.

36. Morrison, L. E. 1988. Time-resolved detection of energy transfer: theory and application to immunoassays. 174(1): 101-20.

37. Tencza S, Islam K, Kalia V, Nasir M, Jolley M, Montelaro R: Development of a fluorescence polarization-based diagnostic assay for equine infectious anemia virus, J Clin Microbiol 2000; 38:1854-9.

38. Schade, S. Z., Jolley, M. E., Sarauer, B. J., Simonson, L. G. (1996) BODIPY-alpha-casein, a pH-independent protein substrate for protease assays using fluorescence polarization. Anal Biochem. 243(1): 1-7.

39. Schade S, Jolley M: Fluorescence polarization assays of enzymes and substrates thereof: Pat. No. 5,804,395. Patent and Trademark Office, United States, Secretary of the Navy, 1998.

40. Nasir M, Jolley M: Fluorescence polarization: An analytical tool for immunoassay and drug discovery, Comb Chem High Throughput Screen 1999; 2:177-90.

41. Nasir M, Jolley M: Fluorescence polarization: Stretching it to its limits. A special edition on fluorescence polarization., Comb Chem High Throughput Screen 2002; in press.

42. Jolley M: Fluorescence polarization immunoassay for the determination of therapeutic drug levels in human plasma, J Anal Toxicology 1981; 5:236-40.

43. Lin M, Sugden E, Jolley M, Stilwell K: Modification of the Mycobacterium bovis extracellular protein MPB70 with fluorescein by fluorescence polarization, Clin Diag Lab Immunol 1996; 3:438-43.

44. Nielsen K, Gall D, Jolley M, et al.: A homogeneous fluorescence polarization assay for detection of antibody to Brucella abortus, J Immunolog Methods 1996; 195:161-8.

Having described the invention, one of skill in the art will appreciate in the appended claims that many modifications and variations of the present invention are possible in light of the above teachings. It is therefore, to be understood that, within the scope of the appended claims, the invention may be practiced otherwise than as specifically described.

1. A competitive method for determining infection wherein determination is by detecting the antigen-specific cytokine induction, in vitro, comprising the steps:
a. collecting a whole blood sample;
b. adding a target agent antigen to said whole blood sample to make an antigen whole blood mixture wherein said target agent is viral, bacteria or protozoa;
c. incubating said whole blood mixture for up to 12 hours;
d. collecting serum or plasma of said whole blood mixture;
e. preparing dilutions of said serum or plasma;
f. adding to said dilutions fluorescently-labeled cytokine probe at about 1 nM;
g. adding to said dilutions cytokine specific antibody;
h. measuring the antibody binding wherein said antibody binding is correlated with cytokine concentration.

2. The competitive method as in claim 1 wherein the method comprises following additional steps:
i. adding said fluorescently-labeled cytokine probe at about 1 nM to dilutions of negative control serum or to dilutions of positive control serum or plasma containing cytokine;
j. adding said cytokine specific antibody to dilutions of said positive and negative control serum containing fluroescently-labeled cytokine probe;
k. measuring the antibody binding in the controls by measuring the change in fluorescence polarization in said positive and negative control dilutions.
l. representing the concentration of the cytokine in said dilutions of positive or negative control serum or plasma, graphically, to obtain a standard curve;
k. comparing the change in fluorescence polarization measured from said serum or plasma with said standard curve to determine the concentration of cytokine.

3. The competitive method as in claim 1 wherein said target agent is Mycobacterium tuberculosis.

4. The competitive method of claim 1, wherein said measuring of said antibody binding is by a method selected from the group consisting of flurorescence polarization, fluorescence lifetime and fluorescence resonance energy transfer.

5. The competitive method as in claim 3, wherein said cytokine is interferon-gamma and said fluorescently-labeled cytokine probe is fluorescently labeled interferon-gamma probe.

6. The competitive method as in claim 1, wherein said cytokine specific antibody is monoclonal or polyclonal.

7. The competitive method as in claim 1, wherein said cytokine probe is a fluorescently-labeled peptide fragment from the cytokine.

8. The competitive method as in claim 5, wherein said interferon-gamma probe is a fluorescent fragment of interferon-gamma.

9. The competitive method as in claim 7, wherein said method can detect cytokine at a concentration of 11 pg/ml or less.

10. A competitive method for determining infection wherein determination is by detecting the dimerization of the antigen-specifically induced cytokine, in vitro, comprising the steps:
a. collecting a whole blood sample;
b. adding a target agent antigen to said whole blood sample to make a antigen whole blood mixture wherein said target agent is viral, bacterial or protozoa;
c. incubating said whole blood mixture for up to 12 hours;
d. collecting serum or plasma of said whole blood mixture;
e. preparing dilutions of said serum or plasma;
f. adding to said dilutions fluorescently-labeled cytokine probe at about 1 nM;
g. measuring dimerization of the cytokine in said dilutions wherein said dimerization is correlated with cytokine concentration.

11. The competitive method of claim 10, wherein said measurement of dimerization is by a method selected from the group consisting of fluorescence polarization, fluorescence lifetime and fluorescence resonance energy transfer.

12. A competitive method as in claim 10 wherein the method comprises following additional steps:
h. adding said fluorescently-labeled cytokine probe at about 1 nM to dilutions of negative control serum or to dilutions of positive control serum or plasma containing cytokine;
i. measuring the dimerization of cytokine in said dilutions of positive or negative control serum;
j. representing the concentration of the cytokine in said dilutions of positive or negative control serum or plasma, graphically, to obtain a standard curve;
k. comparing the change in fluorescence polarization measured from said serum or plasma with said standard curve to determine the concentration of cytokine.

13. A competitive method as in claim 12 wherein said measurement of dimerization is by a method selected from the group consisting of fluorescence polarization, fluorescence lifetime and fluorescence resonance energy transfer

14. A competitive method as in claim 10 wherein said target agent is Mycobacterium tuberculosis.

15. A competitive method as in claim 10, wherein said cytokine is interferon-gamma and said fluorescently-labeled cytokine probe is fluorescently labeled interferon-gamma probe.

16. A competitive method as in claim 10 wherein said cytokine probe is a fluorescent fragment of a cytokine.

17. A competitive method as in claim 14 wherein said interferon-gamma probe is a fluorescent peptide fragment of interferon-gamma.

18. A competitive method as in claim 13, wherein said method can detect cytokine concentration at 11 pg/ml or less.
